(19) United States
(12) Patent Application Publication
(10) Pub. No.: US 2009/0005463 A1
(43) Pub. Date: Jan. 1, 2009

(54) Liquid solubilizers comprising ethoxylated fatty alcohols and ethoxylated triglycerides

(75) Inventors: Wiebke Johannpeter, Steinbach am Taunus (DE); Wibke Stelter, Grosskarben (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant International Ltd.

(21) Appl. No.: 12/215,426

(22) Filed: Jun. 27, 2008

(30) Foreign Application Priority Data
Jun. 29, 2007 (DE) 102007030434.1

(51) Int. Cl. A61K 47/14; C09K 3/00

(52) U.S. Cl. 514/786; 252/182.12

(57) ABSTRACT

Solubilizers are described which comprise
a) one or more ethoxylated fatty alcohols according to formula (I)

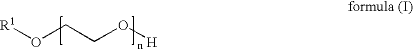

in which R¹ is a linear or branched alkyl radical having 6 to 12 carbon atoms and n is on average a number from 2 to 12,
b) one or more ethoxylated triglycerides whose acyl radicals are derived from linear or branched saturated fatty acids having 6 to 22 carbon atoms, and
c) water
and which are liquid and clear at room temperature and do not comprise addition products of ethylene oxide and simultaneously propylene oxide onto fatty alcohols.

The solubilizers are suitable in particular for producing cosmetic, dermatological or pharmaceutical formulations.

The present invention relates to novel solubilizers with a clear appearance and very good dissolving power of lipophilic substances in compositions with a high water content and their use in cosmetic, dermatological and pharmaceutical formulations.

Lipophilic substances, such as, for example, vitamins, perfume oils or UV photoprotective filters, can often only be incorporated into cosmetic or pharmaceutical preparations with difficulty, particularly if they have a high water content. In such cases, solubilizers, also called hydrotropes, are used. These are individual substances or mixtures with average HLB values which are able to form a bridge from the polar surroundings to the nonpolar substrate. Very effective hydrotropes are the sulfonates of short-chain alkylaromatics, such as, for example, toluene- or cumenesulfonate, although, on account of their inadequate skin-cosmetic compatibility, they are of no importance in the field of cosmetics. Other cosmetic solubilizers, such as, for example, special hydrophilized oils, despite being skin-compatible, do not have adequate dissolving power and/or have poor low-temperature behavior, i.e. they exhibit the tendency to cloud even at room temperature.

US 2004/0043045 describes cosmetic preparations consisting of 40 to 99% by weight of a fatty alcohol having 6 to 22 carbon atoms and 1 to 60% by weight of lipophilic components, for example ethoxylated partial glycerides, and the use of fatty alcohols as solubilizers for active substances with low solubility in water. However, fatty alcohols are unsuitable as solubilizers for producing clear and liquid products.

JP 09301844 discloses that mixtures of ethoxylated octyldodecyl alcohol having 10 to 25 ethylene oxide units and ethoxylated and hydrogenated castor oil having 40 to 80 ethylene oxide units are suitable as solubilizers for lipophilic components in aqueous systems. However, on account of the high degree of ethoxylation of from 10 to 25 and the long fatty alcohol radical of 20 carbon atoms in the ethoxylated octyldodecyl alcohol and the associated relatively high molecular weight, these mixtures also exhibit a relatively high viscosity and accordingly are in need of improvement with regard to handling.

DE 100 25 756 discloses mixtures of a) ethoxylated fatty alcohols, preferably ethoxylated C₁₂-C₁₈-fatty alcohols, b) ethoxylated/propoxylated fatty alcohols and c) ethoxylated triglycerides as solubilizers. These mixtures are liquid, exhibit good dissolving power and are characterized by a clear appearance. However, propoxylated fatty alcohols are not readily biodegradable.

The object of the present invention was thus to provide novel solubilizers which have very good dissolving power, in particular toward lipophilic substances, such as, for example, perfume oils, vitamins, UV photoprotective filters and the like, are liquid and clear at room temperature, have a low-temperature cloud point below 10° C., are toxicologically and ecotoxicologically acceptable and convey a good skin feel.

Surprisingly, it has been found that mixtures of ethoxylated fatty alcohols, characterized by relatively short-chain fatty alcohol radicals having 6 to 12 carbon atoms and an average degree of ethoxylation of 12 or less with ethoxylated triglycerides and water have a very good dissolving power, in particular toward perfume oils, vitamins and UV photoprotective filters, and at the same time are more advantageous than products of the prior art from an ecotoxicological point of view. A further advantage is that the mixtures are liquid and clear at room temperature, can therefore be readily incorporated, impart an aesthetic appearance to the cosmetic products produced using them and additionally have a low-temperature cloud point in the desired range, namely below 10° C.

The invention provides solubilizers comprising
a) one or more ethoxylated fatty alcohols according to formula (I)

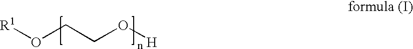

in which R¹ is a linear or branched alkyl radical having 6 to 12, preferably 8 to 12, and particularly preferably 11, carbon atoms and n is on average a number from 2 to 12, preferably 5 to 11, particularly preferably 6 to 9, and especially preferably 8,
b) one or more ethoxylated triglycerides whose acyl radicals are derived from linear or branched saturated fatty acids having 6 to 22 and preferably 12 to 18 carbon atoms, and
c) water,
   wherein the solubilizer is liquid and clear at room temperature and does not contain addition products of ethylene oxide and simultaneously propylene oxide onto fatty alcohols.

In a preferred embodiment of the invention, the solubilizers comprise two or more ethoxylated fatty alcohols according to formula (I), in particular a mixture of linear and branched ethoxylated fatty alcohols, where the weight ratio of ethoxylated fatty alcohols of the formula (I) with linear radicals R¹ to ethoxylated fatty alcohols of the formula (I) with branched radicals R¹ is from 40:60 to 60:40, preferably from 45:55 to 55:45, and particularly preferably 50:50.

In a further preferred embodiment of the invention, the solubilizers comprise one or more ethoxylated fatty alcohols according to formula (I) in which R¹ is a linear or branched alkyl radical having 11 carbon atoms.

In a further preferred embodiment of the invention, the solubilizers comprise one or more ethoxylated fatty alcohols according to formula (I), in which n on average is a number from 6 to 9 and preferably 8.

In a further preferred embodiment of the invention, the solubilizers comprise one or more ethoxylated triglycerides of component b) selected from addition products of, on average, 20 to 100 mol, preferably 30 to 80 mol, particularly preferably 35 to 65 mol and especially preferably 40 mol, of ethylene oxide onto triglycerides, in particular onto hydrogenated castor oil.

The solubilizers according to the invention are liquid and clear and are characterized by low-temperature cloud points below +10° C.

In a further preferred embodiment of the invention, the solubilizers comprise the ethoxylated fatty alcohols of component a) in an amount of from 20 to 70% by weight, preferably from 30 to 65% by weight and particularly preferably from 33 to 60% by weight, the ethoxylated triglycerides of component b) in an amount of from 20 to 60% by weight, preferably from 25 to 55% by weight and particularly preferably from 26 to 51% by weight and water in an amount of from 1 to 20% by weight, preferably from 5 to 18% by weight and particularly preferably from 9 to 16% by weight.

In a particularly preferred embodiment of the invention, the solubilizers are free from components containing propylene oxide.

In an especially preferred embodiment of the invention, the solubilizers consist of the ethoxylated fatty alcohols of component a), the ethoxylated triglycerides of component b) and water.

In a further preferred embodiment of the invention, the liquid solubilizers are characterized by viscosities at 22.5° C. (RV Brookfield viscosimeter, 20 rpm, spindle 3) which are in the range from 100 to 700 mPa·s, preferably in the range from 100 to 600 mPa·s, particularly preferably in the range from 100 to 500 mPa·s.

With the help of the solubilizers according to the invention, it is possible to incorporate components that are sparingly soluble in water, in particular perfume oils, vitamins or UV photoprotective filters, into formulations with high water contents, and to obtain clear products.

The ethoxylated fatty alcohols of component a) are produced industrially by base-catalyzed addition reaction of ethylene oxide onto the primary hydroxyl function of the alcohols.

The addition products of ethylene oxide onto triglycerides which form component b) are known nonionic surfactants. Of suitability are, for example, addition products of, on average, 20 to 100 mol, preferably 30 to 80 mol, particularly preferably 35 to 65 mol, and especially preferably 40 mol, of ethylene oxide onto triglycerides whose acyl radicals are derived from fatty acids having 6 to 22, preferably 12 to 18, carbon atoms. The triglycerides may be synthetic in nature. However, they are preferably natural, especially vegetable, fats and oils which are reacted with ethylene oxide following refinement and hardening. Ethoxylation can take place here by inserting ethylene oxide into the carbonyl ester group. However, addition onto secondary hydroxyl groups present in the molecule is easier and therefore also preferred, for which reason hardened ricinus oil (castor oil) is particularly preferred as starting material. From an applications point of view, the use of addition products of, on average, 35 to 65 mol and preferably 40 mol, of ethylene oxide onto hardened, i.e. hydrogenated, castor oil, in particular, is advisable.

The solubilizers according to the invention can serve for the production of cosmetic, dermatological or pharmaceutical formulations, such as, for example, hair shampoos, hair lotions, foam baths, shower baths, oral and dental care compositions, creams, gels, lotions, alcoholic and aqueous/alcoholic solutions, emulsions, wax/fat masses, stick preparations, powders or ointments.

The present invention therefore also further provides the use of a solubilizer according to the invention for producing cosmetic, dermatological and pharmaceutical formulations.

The present invention also further provides cosmetic, dermatological or pharmaceutical formulations produced using a solubilizer according to the invention.

In these cosmetic, dermatological or pharmaceutical formulations, the solubilizers according to the invention are preferably present in amounts of from 0.1 to 10.0% by weight, based on the finished formulations.

These formulations according to the invention comprise either no substances containing propylene oxide or if they do comprise substances containing propylene oxide, comprise, based on the finished formulations, preferably 1.0% by weight or

less, particularly preferably 0.05% by weight or less, and especially preferably 0.01% by weight or less, of substances containing propylene oxide.

The cosmetic, dermatological or pharmaceutical formulations according to the invention produced using the solubilizers according to the invention particularly preferably comprise
a1) C₁₁-alkyl-O—(CH₂CH₂O)₈—H,
b1) ethoxylated hydrogenated castor oil with 40 ethylene oxide units, and
c) water and
d) either do not comprise substances containing propylene oxide or if they do comprise substances containing propylene oxide, comprise, based on the finished formulations, 1.0% by weight or less, preferably 0.05% by weight or less and particularly preferably 0.01% by weight or less, of substances containing propylene oxide.

Since such cosmetic, dermatological or pharmaceutical formulations can also be produced using the individual components present in the solubilizers according to the invention instead of using the solubilizers according to the invention, the present invention also further provides cosmetic, dermatological or pharmaceutical formulations which comprise
a1) C₁₁-alkyl-O—(CH₂CH₂O)₈—H,
b1) ethoxylated hydrogenated castor oil with 40 ethylene oxide units, and
c) water and
d) either do not comprise substances containing propylene oxide or if they do comprise substances containing propylene oxide, comprise, based on the finished formulations, 1.0% by weight or less, preferably 0.05% by weight or less and particularly preferably 0.01% by weight or less, of substances containing propylene oxide.

Preferably, based on the finished formulations, the ethoxylated alcohol C₁₁-alkyl-O—(CH₂CH₂O)₈—H of component a1) is present from 0.05 to 6.0% by weight and the ethoxylated hydrogenated castor oil having 40 ethylene oxide units of component b1) is present from 0.02 to 5.0% by weight in the formulations according to the invention.

Particularly preferably, based on the finished formulations, the ethoxylated alcohol C₁₁-alkyl-O—(CH₂CH₂O)₈—H of component a1) is present from 0.056 to 5.6% by weight and the ethoxylated hydrogenated castor oil having 40 ethylene oxide units of component b1) is present from 0.03 to 3.0% by weight in the formulations according to the invention.

In an especially preferred embodiment of the invention, the formulations according to the invention do not comprise substances containing propylene oxide.

The formulations according to the invention comprise, based on the finished formulations, preferably an amount of water of 30.0% by weight or more, particularly preferably from 40.0 to 95.0% by weight, especially preferably from 50.0 to 95.0% by weight and extraordinarily preferably from 60.0 to 95.0% by weight.

Furthermore, the formulations according to the invention preferably comprise components that are sparingly soluble in water, in particular selected from perfume oils, vitamins and UV photoprotective filters. These formulations according to the invention comprise, based on the finished formulations, preferably 0.01 to 30.0% by weight, particularly preferably 0.01 to 20.0% by weight, especially preferably 0.05 to 10.0% by weight and extraordinarily preferably 0.5 to 5.0% by weight, of components that are sparingly soluble in water, in particular of components that are sparingly soluble in water selected from perfume oils, vitamins and UV photoprotective filters.

In a particularly preferred embodiment of the invention, the formulations according to the invention comprise one or more perfume oils that are sparingly soluble in water. The perfume oils that are sparingly soluble in water are present in the formulations according to the invention in amounts of from preferably 0.01 to 20.0% by weight, particularly preferably 0.05 to 10.0% by weight and especially preferably 0.5 to 5.0% by weight, based on the finished formulations according to the invention.

Available as perfume oils that are sparingly soluble in water are mixtures of natural and synthetic fragrances. Preferred natural fragrances are extracts of flowers (roses, lilies, lavender, chamomile, linden blossom, jasmine, neroli, ylang-ylang), stems and leaves (geranium, patchouli, petit grain), fruits (anise, cloves, coriander, caraway, juniper, mango, peach, vanilla), fruit peels (bergamot, lemons, oranges), roots (mace, angelica, celery, cardamom, costus, iris, calmus), woods (pinewood, sandalwood, guaic wood, cedar wood, rosewood), herbs and grasses (tarragon, lemon grass, sage, thyme, rosemary, mint, melissa, cinnamon leaf, needles and branches (spruce, fir, pine, dwarf-pine), resins and balsams (galbanum, elemi, benzoe, myrrh, olibanum, opoponax). Furthermore preferred scent or perfume oils that may be used are also synthetic products of the ester type, ether type, aldehyde type, ketone type, alcohol type and hydrocarbon type. Fragrance compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl methylphenylglycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroxycitronellal, lilial and bourgeonal, the ketones include, for example, the ionones, alpha-isomethylionone and methyl cedryl ketone, the alcohols include anethole, citronellol, eugenol, geraniol, linalool, phenylethyl alcohol and terpineol, the hydrocarbons include primarily the terpenes and balsams. Preference is given to using mixtures of different fragrances which together produce a pleasant scent note.

In a further particularly preferred embodiment of the invention, the formulations according to the invention comprise one or more vitamins that are sparingly soluble in water. The vitamins that are sparingly soluble in water are present in the formulations according to the invention in amounts of from preferably 0.001 to 10.0% by weight, particularly preferably 0.01 to 10.0% by weight, especially preferably 0.05 to 10.0% by weight and extraordinarily preferably 0.5 to 5.0% by weight, based on the finished formulations according to the invention.

Vitamins that are sparingly soluble in water that can preferably be used are vitamin A, vitamin A derivatives, carotenes or derivatives thereof, ascorbic acid (vitamin C), and also tocopherol (vitamin E) and/or derivatives thereof.

In a further particularly preferred embodiment of the invention, the formulations according to the invention comprise one or more UV filters that are sparingly soluble in water. The UV filters that are sparingly soluble in water are present in the formulations according to the invention in amounts of from preferably 0.01 to 20.0% by weight, particularly preferably 0.05 to 10.0% by weight and especially preferably 0.5 to 5.0% by weight, based on the finished formulations according to the invention.

Suitable UV filters that are sparingly soluble in water are preferably 4-aminobenzoic acid; 3-(4′-trimethylammonium)benzylidenebornan-2-one methylsulfate; 3,3,5-trimethylcyclohexyl salicylate; 2-hydroxy-4-methoxybenzophenone; 2-phenylbenzimidazole-5-sulfonic acid and its potassium, sodium and triethanolamine salts; 3,3′-(1,4-phenylenedimethine)bis(7,7-dimethyl-2-oxobicyclo[2.2.1]heptane-1-methanesulfonic acid) and its salts; 1-(4-tert-butylphenyl)-3-(4-methoxyphenyl)propane-1,3-dione, 3-(4′-sulfo)benzylidenebornan-2-one and its salts; 2-ethylhexyl 2-cyano-3,3-diphenylacrylate; polymer of N-[2(and 4)(2-oxoborn-3-ylidenemethyl)benzyl]acrylamide; 2-ethylhexyl 4-methoxycinnamate; ethoxylated ethyl 4-aminobenzoate; isoamyl 4-methoxycinnamate; 2,4,6-tris[p-(2-ethylhexyloxycarbonyl)anilino]-1,3,5-triazine; 2-(2H-benzotriazol-2-yl)-4-methyl-6-(2-methyl-3-(1,3,3,3-tetramethyl-1-(trimethylsilyloxy)disiloxanyl)propyl)phenol; bis(2-ethylhexyl) 4,4′-[(6-[4-((1,1-dimethylethyl)aminocarbonyl)phenylamino]-1,3,5-triazin-2,4-yl)diimino]bisbenzoate; 3-(4′-methylbenzylidene)-D,L-camphor; 3-benzylidenecamphor; 2-ethylhexyl salicylate; 2-ethylhexyl 4-dimethylaminobenzoate; hydroxy-4-methoxybenzophenone-5-sulfonic acid (sulisobenzonum) and the sodium salt; and/or 4-isopropylbenzyl salicylate.

The cosmetic, dermatological or pharmaceutical formulations according to the invention can comprise, as further auxiliaries and additives, surfactants, emulsifiers, cationic polymers, thickeners, film formers, antimicrobial active ingredients, astringents, antioxidants, UV photoprotective filters, pigments/micropigments, gelling agents, and further additives customary in cosmetics, dermatology or pharmacy, such as, for example, superfatting agents, moisturizing agents, silicones, stabilizers, conditioners, glycerol, preservatives, pearlizing agents, dyes, enzyme inhibitors, solvents, hydrotropes, opacifiers, fatty alcohols, substances with keratolytic and keratoplastic effect, antidandruff agents, biogenic active ingredients (local anesthetics, antibiotics, antiphlogistic, antiallergics, corticosteroids, sebostatics), vitamins, Bisabolol®, allantoin, Phytantriol®, Panthenol®, AHA acids, plant extracts, for example aloe vera and proteins.

Where substances are specified below as further auxiliaries and additives for the cosmetic, dermatological or pharmaceutical formulations according to the invention that are already encompassed by the solubilizers according to the invention, either explicitly as individual compounds or in the form of generic expressions, this is because these substances can in principle be used in addition to the solubilizers according to the invention for producing the formulations according to the invention.

In a preferred embodiment of the invention, the group of further auxiliaries and additives specified below, however, does not include the compounds of components a) and b) of the solubilizers according to the invention. In a further preferred embodiment of the invention, the group of further auxiliaries and additives specified below only does not include the substances C₁₁-alkyl-O—(CH₂CH₂O)₈—H and ethoxylated hydrogenated castor oil with 40 ethylene oxide units.

Anionic washing-active substances or surfactants that may be mentioned are preferably: C₁₀-C₂₀-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfates, alkanesulfonates and hydroxyalkanesulfonates, olefin sulfonates, acyl esters of isethionates, α-sulfo fatty acid esters, alkylbenzenesulfonates, alkylphenol glycol ether sulfonates, sulfosuccinates, sulfosuccinic half-esters and diesters, fatty alcohol ether phosphates, protein-fatty acid condensation products, alkyl monoglyceride sulfates and sulfonates, alkyl glyceride ether sulfonates, fatty acid methyl taurides, fatty acid sarcosinates, sulfo ricinoleates, amphoacetates or -glycinates, acylglutamates. These compounds and mixtures thereof are used in the form of their water-soluble or water-dispersible salts, for example the sodium, potassium, magnesium, ammonium, mono-, di- and triethanolammonium salts and also the analogous alkylammonium salts.

The amount of anionic surfactants in the formulations according to the invention is preferably 1 to 30% by weight, particularly preferably 5 to 25% by weight and especially preferably 10 to 22% by weight, based on the finished formulations.

Suitable cationic surfactants are, for example, quaternary ammonium salts, such as di(C₁₀-C₂₄-alkyl)dimethylammonium chloride or bromide, preferably di(C₁₂-C₁₈-alkyl)dimethylammonium chloride or bromide; C₁₀-C₂₄-alkyldimethylethylammonium chloride or bromide; C₁₀-C₂₄-alkyltrimethylammonium chloride or bromide, preferably cetyltrimethylammonium chloride or bromide and C₂₀-C₂₂-alkyltrimethylammonium chloride or bromide; C₁₀-C₂₄-alkyldimethylbenzylammonium chloride or bromide, preferably C₁₂-C₁₈-alkyldimethylbenzylammonium chloride; N—(C₁₀-C₁₈-alkyl)pyridinium chloride or bromide, preferably N—(C₁₂-C₁₆-alkyl)pyridinium chloride or bromide; N—(C₁₀-C₁₈-alkyl)isoquinolinium chloride, bromide or monoalkyl sulfate; N—(C₁₂-C₁₈-alkylpolyoylaminoformylmethyl)pyridinium chloride; N—(C₁₂-C₁₈-alkyl)-N-methylmorpholinium chloride, bromide or monoalkyl sulfate; N—(C₁₂-C₁₈-alkyl)-N-ethylmorpholinium chloride, bromide or monoalkyl sulfate; C₁₆-C₁₈-alkylpentaoxyethylammonium chloride; diisobutylphenoxyethoxyethyldimethylbenzylammonium chloride; salts of N,N-diethylaminoethylstearylamide and -oleylamide with hydrochloric acid, acetic acid, lactic acid, citric acid, phosphoric acid; N-acylaminoethyl-N,N-diethyl-N-methylammonium chloride, bromide or monoalkyl sulfate and N-acylaminoethyl-N,N-diethyl-N-benzylammonium chloride, bromide or monoalkyl sulfate, where acyl is preferably stearyl or oleyl.

The amount of cationic surfactants in the formulations according to the invention is preferably 0.1 to 10% by weight, particularly preferably 0.2 to 7% by weight, and especially preferably 0.5 to 5% by weight, based on the finished formulations.

Suitable nonionic surfactants which can be used as washing-active substances are preferably fatty alcohol ethoxylates (alkyl polyethylene glycols); alkylphenol polyethylene glycols; alkyl mercaptan polyethylene glycols; fatty amine ethoxylates (alkylaminopolyethylene glycols); fatty acid ethoxylates (acyl polyethylene glycols); polypropylene glycol ethoxylates (Pluronics®); fatty acid amidepolyethylene glycols; N-alkyl-, N-alkoxypolyhydroxy fatty acid amide, in particular fatty acid N-methyl glucamides, sucrose ester; polyglycol ether, alkyl polyglycosides, phosphoric acid esters (mono-, di- and triphosphoric acid esters ethoxylated and non-ethoxylated).

The amount of nonionic surfactants in the formulations according to the invention (e.g. in the case of rinse-off products) is preferably in the range from 1 to 20% by weight, particularly preferably 2 to 10% by weight, and especially preferably 3 to 7% by weight, based on the finished formulations.

Preferred amphoteric surfactants are: N—(C₁₂-C₁₈-alkyl)-β-aminopropionates and N—(C₁₂-C₁₈-alkyl)-β-iminodipropionates as alkali metal and mono-, di- and trialkylammonium salts; N-acylaminoalkyl-N,N-dimethylacetobetaine, preferably N—(C₈-C₁₈-acyl)aminopropyl-N,N-dimethylacetobetaine; C₁₂-C₁₈-alkyldimethylsulfopropylbetaine; amphoteric surfactants based on imidazoline (trade name: Miranol®, Steinapon®), preferably the sodium salt of 1-(β-carboxymethyloxyethyl)-1-(carboxymethyl)-2-laurylimidazolinium; amine oxides, e.g. C₁₂-C₁₈-alkyldimethylamine oxide, fatty acid amidoalkyldimethylamine oxide.

The amount of amphoteric surfactants in the formulations according to the invention is preferably 0.5 to 20% by weight and particularly preferably 1 to 10% by weight, based on the finished formulations.

Furthermore, foam-boosting cosurfactants from the group alkylbetaines, alkylamidobetaines, aminopropionates, aminoglycinates, imidazoliniumbetaines and sulfobetaines, amine oxides and fatty acid alkanolamides or polyhydroxyamides can be used in the formulations according to the invention.

Preferred surfactants in the formulations according to the invention are alkyl ether sulfates, alkyl sulfates, in particular lauryl sulfate, alkylbetaines, alkylamidopropylbetaines, in particular cocoamidopropylbetaine, amphoacetates, acylglutamates, in particular sodium cocoylglutamate, alkyl ether sulfosuccinates, in particular disodium laureth sulfosuccinate, and coconut fatty acid diethanolamide.

The total amount of surfactants used in the formulations according to the invention is preferably 1 to 70% by weight, particularly preferably 10 to 40% by weight, and especially preferably 12 to 35% by weight, based on the finished formulations.

Formulations according to the invention present as emulsions can be produced without further emulsifier or else comprise one or more emulsifiers. These emulsifiers can be selected from the group of nonionic, anionic, cationic or amphoteric emulsifiers.

Suitable nonionogenic coemulsifiers are preferably addition products of from 0 to 30 mol of ethylene oxide and/or 0 to 5 mol of propylene oxide onto linear fatty alcohols having 8 to 22 carbon atoms, onto fatty acids having 12 to 22 carbon atoms,

onto alkylphenols having 8 to 15 carbon atoms in the alkyl group and onto sorbitan or sorbitol esters; (C₁₂-C₁₈)-fatty acid mono- and diesters of addition products of from 0 to 30 mol of ethylene oxide onto glycerol; glycerol mono- and diesters and sorbitan mono- and diesters of saturated or unsaturated fatty acids having 6 to 22 carbon atoms and optionally ethylene oxide addition products thereof; addition products of from 15 to 60 mol of ethylene oxide onto castor oil and/or hydrogenated castor oil; polyol and in particular polyglycerol esters, such as, for example, polyglycerol polyricinoleate and polyglycerol poly-12-hydroxystearate. Likewise suitable are preferably ethoxylated fatty amines, fatty acid amides, fatty acid alkanolamides and mixtures of compounds from two or more of these substance classes.

Suitable ionogenic coemulsifiers are, for example, anionic emulsifiers, such as mono-, di- or triphosphoric acid esters, soaps (e.g. sodium stearate), fatty alcohol sulfates, but also cationic emulsifiers, such as mono-, di- and trialkyl quats and polymeric derivatives thereof.

Available amphoteric emulsifiers are preferably alkylaminoalkylcarboxylic acids, betaines, sulfobetaines and imidazoline derivatives.

Furthermore, naturally occurring emulsifiers can be used, among which beeswax, wool wax, lecithin and sterols are preferred.

Preferably, fatty alcohol ethoxylates are selected from the group of ethoxylated stearyl alcohols, cetyl alcohols, oleyl alcohols, lauryl alcohols, cetylstearyl alcohols, in particular polyethylene glycol(13) stearyl ether, polyethylene glycol(14) stearyl ether, polyethylene glycol(15) stearyl ether, polyethylene glycol(16) stearyl ether, polyethylene glycol(17) stearyl ether, polyethylene glycol(18) stearyl ether, polyethylene glycol(19) stearyl ether, polyethylene glycol(20) stearyl ether,

polyethylene glycol(12) isostearyl ether, polyethylene glycol(13) isostearyl ether, polyethylene glycol(14) isostearyl ether, polyethylene glycol(15) isostearyl ether,
polyethylene glycol(16) isostearyl ether, polyethylene glycol(17) isostearyl ether,
polyethylene glycol(18) isostearyl ether, polyethylene glycol(19) isostearyl ether, polyethylene glycol(20) isostearyl ether, polyethylene glycol(13) cetyl ether, polyethylene glycol(14) cetyl ether, polyethylene glycol(15) cetyl ether, polyethylene glycol(16) cetyl ether, polyethylene glycol(17) cetyl ether, polyethylene glycol(18) cetyl ether, polyethylene glycol(19) cetyl ether, polyethylene glycol(20) cetyl ether, polyethylene glycol(13) isocetyl ether, polyethylene glycol(14) isocetyl ether, polyethylene glycol(15) isocetyl ether, polyethylene glycol(16) isocetyl ether, polyethylene glycol(17) isocetyl ether, polyethylene glycol(18) isocetyl ether, polyethylene glycol(19) isocetyl ether, polyethylene glycol(20) isocetyl ether, polyethylene glycol(12) oleyl ether, polyethylene glycol(13) oleyl ether, polyethylene glycol(14) oleyl ether, polyethylene glycol(15) oleyl ether, polyethylene glycol(12) lauryl ether, polyethylene glycol(12) isolauryl ether, polyethylene glycol(13) cetylstearyl ether, polyethylene glycol(14) cetylstearyl ether, polyethylene glycol(15) cetylstearyl ether, polyethylene glycol(16) cetylstearyl ether, polyethylene glycol(17) cetylstearyl ether, polyethylene glycol(18) cetylstearyl ether, polyethylene glycol(19) cetylstearyl ether, polyethylene glycol(20) cetylstearyl ether.

Fatty acid ethoxylates are preferably selected from the group of ethoxylated stearates, isostearates and oleates, in particular polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20) oleate.

An ethoxylated alkyl ether carboxylic acid or salts thereof that can be used is advantageously sodium laureth-11 carboxylate.

Ethoxylated triglycerides that can be used are advantageously polyethylene glycol(60) evening primrose glycerides.

Furthermore, it is advantageous to select the polyethylene glycol glycerol fatty acid esters from the group polyethylene glycol(20) glyceryl laurate, polyethylene glycol(6) glyceryl caprate/caprinate, polyethylene glycol(20) glyceryl oleate, polyethylene glycol(20) glyceryl isostearate and polyethylene glycol(18) glyceryl oleate/cocoate.

Among the sorbitan esters, polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate in particular are suitable.

Advantageous W/O emulsifiers that can be used are: fatty alcohols having 8 to 30 carbon atoms, monoglycerol esters of saturated and/or unsaturated, branched and/or unbranched alkanecarboxylic acids of chain length from 8 to 24, in particular 12 to 18, carbon atoms, diglycerol esters of saturated and/or unsaturated, branched and/or unbranched alkanecarboxylic acids of chain length from 8 to 24, in particular 12 to 18, carbon atoms, monoglycerol ethers of saturated and/or unsaturated, branched and/or unbranched alcohols of chain length from 8 to 24, in particular 12 to 18, carbon atoms, diglycerol ethers of saturated and/or unsaturated, branched and/or unbranched alcohols of chain length from 8 to 24, in particular 12 to 18, carbon atoms, propylene glycol esters of saturated and/or unsaturated, branched and/or unbranched alkanecarboxylic acids of chain length from 8 to 24, in particular 12 to 18, carbon atoms, and also sorbitan esters of saturated and/or unsaturated, branched and/or unbranched alkanecarboxylic acids of chain length from 8 to 24, in particular 12 to 18, carbon atoms. Particularly advantageous W/O emulsifiers are glyceryl monostearate, glyceryl monoisostearate, glyceryl monomyristate, glyceryl monooleate, glyceryl monolaurate, glyceryl monocaprylate, glyceryl monocaprinate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol or polyethylene glycol(2) stearyl ether.

The amount of emulsifier or emulsifiers present in the formulations according to the invention is preferably 0.1 to 20% by weight, particularly preferably 0.5 to 15% by weight, and especially preferably 1 to 10% by weight, based on the finished formulations.

Suitable cationic polymers are preferably the compounds known under the INCI name “polyquaternium”, in particular polyquaternium-31, polyquaternium-16, polyquaternium-24, polyquaternium-7, polyquaternium-22, polyquaternium-39, polyquaternium-28, polyquaternium-2, polyquaternium-10, polyquaternium-11, polyquaternium 37&mineral oil&PPG trideceth (Salcaree® SC95), PVP-dimethylaminoethyl methacrylate copolymer, guar hydroxypropyltriammonium chlorides, and calcium alginate and ammonium alginate.

Furthermore, use may preferably be made of cationic cellulose derivatives; cationic starch; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethyleneimines; cationic silicone polymers, such as, for example, amidomethicones; copolymers of adipic acid and dimethylaminohydroxy-propyldiethylenetriamine; polyaminopolyamide and cationic chitin derivatives, such as, for example, chitosan.

The amount of cationic polymers in the formulations according to the invention can preferably be in the range from 0.1 to 10% by weight, particularly preferably in the range from 0.2 to 5% by weight, and especially preferably in the range from 0.5 to 2.5% by weight, based on the finished formulations.

The desired viscosity of the formulations can be adjusted by adding thickeners. Of suitability are preferably cellulose ethers and other cellulose derivatives (e.g. carboxymethylcellulose, hydroxyethylcellulose), gelatin, starch and starch derivatives, sodium alginates, fatty acid polyethylene glycol esters, agar agar, tragacanth or dextrin derivatives, in particular dextrin esters.

Various materials are used as synthetic polymers, preferably polyvinyl alcohols, polyacrylamides, polyvinylamides, polysulfonic acids, in particular copolymers based on ammonium salts of acrylamidoalkylsulfonic acids and cyclic N-vinylcarboxamides or cyclic and linear N-vinylcarboxamides or else hydrophobically modified acrylamidoalkylsulfonic acid copolymers, polyacrylic acid, polyacrylic acid derivatives, polyacrylic acid esters, polyvinylpyrrolidone, polyvinyl methyl ether, polyethylene oxides, copolymers of maleic anhydride and vinyl methyl ether, and various mixtures and copolymers of the above-mentioned compounds, including their various salts and esters. These polymers can be crosslinked or uncrosslinked.

Thickeners of particular suitability especially for oil-based formulations are dextrin esters, for example dextrin palmitate, but also fatty acid soaps, fatty alcohols and silicone waxes, for example alkylmethicones, SilCare® 41M40, SilCare® 41M50, SilCare® 41M65, SilCare® 41M70 or SilCare® 41M80.

Depending on the intended use, preferred film formers are salts of phenylbenzimidazolesulfonic acid, water-soluble polyurethanes, for example C₁₀-polycarbamylpolyglyceryl ester, polyvinyl alcohol, polyvinylpyrrolidone copolymers, for example vinylpyrrolidone/vinyl acetate copolymer, water-soluble acrylic acid polymers/copolymers or esters or salts thereof, for example partial ester copolymers of acrylic/methacrylic acid and polyethylene glycol ethers of fatty alcohols, such as acrylate/steareth-20 methacrylate copolymer, water-soluble cellulose, for example hydroxymethylcellulose, hydroxyethylcellulose, hydroxypropylcellulose, water-soluble quaternium, polyquaternium, carboxyvinyl polymers, such as carbomers and salts thereof, polysaccharides, for example polydextrose and glucan, vinyl acetate/crotonate, available for example under the trade name Aristoflex® A 60 (Clariant), and also polymeric amine oxides, for example representatives available under the trade names Diaformer Z-711, 712, 731, 751.

Preferably suitable antimicrobial active ingredients are cetyltrimethylammonium chloride, cetylpyridinium chloride, diisobutylethoxyethyldimethylbenzylammonium chloride, sodium N-laurylsarcosinate, sodium N-palmethylsarcosinate, lauroylsarcosine, N-myristoylglycine, potassium N-laurylsarcosine, trimethylammonium chloride, sodium aluminum chlorohydroxylactate, triethyl citrate, tricetylmethylammonium chloride, 2,4,4′-trichloro-2′-hydroxydiphenyl ether (triclosan), phenoxyethanol, 1,5-pentanediol, 1,6-hexanediol, 3,4,4′-trichlorocarbanilide (triclocarban), diaminoalkylamide, for example L-lysine hexadecylamide, citrate heavy metal salts, salicylates, piroctose, in particular zinc salts, pyrithiones and heavy metal salts thereof, in particular zinc pyrithione, zinc phenolsulfate, farnesol and combinations of these active substances.

The formulations according to the invention comprise the antimicrobial agents preferably in amounts up to 50% by weight, particularly preferably in amounts of from 0.01 to 10% by weight, and especially preferably in amounts of from 0.1 to 10% by weight, based on the finished formulations.

Preferred astringents are oxides, preferably magnesium oxide, aluminum oxide, titanium dioxide, zirconium dioxide and zinc oxide, oxide hydrates, preferably aluminum oxide hydrate (boehmite) and hydroxides, preferably of calcium, magnesium, aluminum, titanium, zirconium or zinc.

The formulations according to the invention comprise the astringent active ingredients preferably in amounts of from 0 to 50% by weight, particularly preferably in amounts of from 0.01 to 10% by weight and especially preferably in amounts of from 0.1 to 10% by weight, based on the finished formulations.

Advantageous formulations according to the invention comprise one or more antioxidants.

Favorable but nevertheless optional antioxidants to be used may be any antioxidants that are suitable or customary for cosmetic, dermatological and/or pharmaceutical applications.

The antioxidants are advantageously selected from the group consisting of amino acids (e.g. glycine, histidine, tyrosine, tryptophane) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. (α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxine, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and also salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, penta-, hexa-, heptathionine sulfoximine) in very low tolerated doses (e.g. pmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg-ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide), superoxide dismutase and the derivatives (salts, esters, ethers, sugars, nucleotides, nucleosides, peptides and lipids) suitable according to the invention of these specified substances.

For the purposes of the present invention, water-soluble antioxidants can be used particularly advantageously.

The antioxidants can protect the skin and the hair against oxidative stress. Preferred antioxidants here are vitamin E and derivatives thereof, and vitamin A and derivatives thereof.

The amount of antioxidants (one or more compounds) in the formulations according to the invention is preferably 0.001 to 30% by weight, particularly preferably 0.05 to 20% by weight, and in particular 1 to 10% by weight, based on the finished formulations.

Pigments/micropigments that can be used are preferably microfine titanium dioxide, mica-titanium oxide, iron oxides, mica-iron oxide, zinc oxide, silicon oxides, ultramarine blue, chromium oxides.

Suitable gelling agents are all surface-active substances which form a network structure dissolved in the liquid phase and thus solidify the liquid phase.

Suitable gelling agents are specified, for example, in WO 98/58625.

Preferred gelling agents are metal salts of fatty acids, preferably having 12 to 22 carbon atoms, for example sodium stearate, sodium palmitate, sodium laurate, sodium arachidates, sodium behenate, potassium stearate, potassium palmitate, sodium myristate, aluminum monostearate, hydroxy fatty acids, for example 12-hydroxystearic acid, 16-hydroxyhexadecanoyl acid; fatty acid amides; fatty acid alkanolamides; dibenzalsorbitol and alcohol-soluble polyamides and polyacrylamides or mixtures thereof.

Preferably, the formulations according to the invention comprise 0.01 to 20% by weight, particularly preferably 0.1 to 10% by weight, especially preferably 1 to 8% by weight and extraordinarily preferably 3 to 7% by weight, of gelling agents, based on the finished formulations.

Further additives may be silicone compounds, preferably dimethylpolysiloxane, methylphenylpolysiloxanes, cyclic silicones, and amino-, fatty-acid-, alcohol-, polyether-, epoxy-, fluorine- and/or alkyl-modified silicone compounds, for example alkylsilicones SilCare® Silicone 41M10, SilCare® Silicone 41M15, SilCare® Silicone 41M20, SilCare® Silicone 41M30 (Clariant), alkyltrimethicones SilCare® 31M30, SilCare® 31M40, SilCare® 31M50, SilCare® 31M60 (Clariant), phenyltrimethicones SilCare® 15M30, SilCare® 15M40, SilCare® 15M50, SilCare® 15M60 (Clariant), polyalkylarylsiloxanes and polyether siloxane copolymers.

The formulations according to the invention can comprise the abovementioned silicone compounds preferably in the amounts from 0.1 to 20% by weight, particularly preferably 0.2 to 15% by weight, especially preferably 0.5 to 10% by weight, based on the finished formulations.

Suitable carrier materials are preferably vegetable oils, natural and hardened oils, waxes, fats, water, alcohols, polyols, glycerol, glycerides, liquid paraffins, liquid fatty alcohols, sterol, polyethylene glycols, cellulose and cellulose derivatives.

Fungicidal active ingredients that can be used are preferably ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, Zn pyrithione and Octopirox® in the amounts of from 0.05 to 5% by weight, preferably 0.1 to 3% by weight, and especially preferably 0.2 to 2% by weight, based on the finished formulations.

The formulations according to the invention can advantageously be mixed with conventional ceramides, pseudoceramides, fatty acid N-alkylpolyhydroxyalkylamides,

cholesterol, cholesterol fatty acid esters, fatty acids, triglycerides, cerebrosides, phospholipids and similar substances.

As pearlizing compounds, preference is given to fatty acid monoalkanolamides, fatty acid dialkanolamides, monoesters or diesters of alkylene glycol, in particular of ethylene glycol and/or propylene glycol or oligomers thereof with higher fatty acids, e.g. palmitic acid, stearic acid or behenic acid or mixtures thereof, mono- or diesters of alkylene glycols with fatty acids, fatty acids and metal salts thereof, monoesters or polyesters of glycerol with carboxylic acids and ketosulfones of various types. Ethylene glycol distearate and polyethylene glycol distearate with 3 glycol units are particularly preferred as pearlizing component in the formulations according to the invention.

As moisturizing substance, preferably isopropyl palmitate, glycerol and/or sorbitol are available, these preferably being used in amounts of from 0.1 to 50% by weight, based on the finished formulations according to the invention.

Superfatting agents which can be used are preferably lanolin and lecithin, nonethoxylated and polyethoxylated or acylated lanolin and lecithin derivatives, polyol fatty acid esters, mono-, di- and triglycerides and/or fatty acid alkanolamides.

Suitable preservatives are preferably phenoxyethanol, parabens, pentanediol or sorbic acid. They are used preferably in amounts of from 0.001 to 5% by weight, particularly preferably from 0.01 to 3% by weight, and especially preferably from 0.1 to 2% by weight, based on the finished formulations according to the invention.

Suitable enzyme inhibitors are, for example, esterase inhibitors. These are preferably trialkyl citrates, such as trimethyl citrate, tripropyl citrate, triisopropyl citrate, tributyl citrate and in particular triethyl citrate (Hydagen™ CAT, Henkel KGaA). The substances inhibit the enzyme activity and thereby reduce the formation of odor. Further substances which are contemplated as esterase inhibitors are sterol sulfates or phosphates, such as, for example, lanosterol, cholesterol, campesterol, stigmasterol and sitosterol sulfate or phosphate, dicarboxylic acids and esters thereof, such as, for example, glutaric acid, monoethyl glutarate, diethyl glutarate, adipic acid, monoethyl adipate, diethyl adipate, malonic acid and diethyl malonate, hydroxycarboxylic acids and esters thereof, such as, for example, citric acid, malic acid, tartaric acid or diethyl tartrate, and also zinc glycinate.

Suitable odor absorbers are substances which can absorb and largely retain odor-forming compounds. They reduce the partial pressure of the individual components and thus also reduce their rate of spread. It is important that here perfumes must remain unaffected. Odor absorbers have no effectiveness against bacteria. They comprise, for example, as main constituent, a complex zinc salt of ricinoleic acid or special, largely odor-neutral fragrances which are known to the person skilled in the art as “fixatives”, such as, for example, extracts of labdanum or styrax or certain abietic acid derivatives.

As dyes, the substances approved and suitable for cosmetic, dermatological or pharmaceutical purposes can be used.

The total fraction of auxiliaries and additives can be 1 to 50% by weight and preferably 5 to 40% by weight, based on the finished formulations according to the invention. The preparation of the formulations according to the invention can take place by customary cold or hot processes.

The acids or alkalis used for adjusting the pH are preferably mineral acids, in particular HCl, inorganic bases, in particular NaOH or KOH, and organic acids, in particular citric acid.

The formulations according to the invention are adjusted preferably to a pH in the range 2 to 12 and particularly preferably to a pH in the range 3 to 8.

In a further particularly preferred embodiment of the invention, the formulations according to the invention are formulations with a clear appearance.

The examples and applications below are intended to illustrate the invention in more detail but without limiting it thereto (all of the percentages given are percent by weight; % by weight).

### EXAMPLES

### Examples 1 to 4

### Solubilizers

| | | |
|---|---|---|
| 1 | Genapol ® UD 088 | 50% by wt. |
| | Emulsogen ® HCO 040 | 50% by wt. |
| 2 | Genapol ® UD 088 | 50% by wt. |
| | Emulsogen ® HCO 060 | 50% by wt. |
| 3 | Genapol ® UD 088 | 70% by wt. |
| | Emulsogen ® HCO 040 | 30% by wt. |
| 4 | Genapol ® UD 110 | 44% by wt. |
| | Emulsogen ® HCO 060 | 44% by wt. |
| | Water | 12% by wt. |

Examples 1 to 4 are in accordance with the invention. Examples 1 to 3 are, for example, therefore in accordance with the invention because Genapol® UD 088 comprises 20% by weight of water.

### Formulation Examples 5 to 7

### Aftershave Gel

Composition:

| | | |
|---|---|---|
| A | Solubilizer as in Example 1, 2 or 3 | 1.5% |
| B | Vitamin E | 0.2% |
| | Menthol | 0.2% |
| C | Alcohol | 30.0% |
| D | Water | ad 100% |
| | Allantoin | 0.2% |
| | Polyglycol 400 | 3.0% |
| | Polyglycol 35000 P | 1.0% |
| E | Aristoflex ® AVC | 1.0% |

Preparation:
I Components A and B are initially introduced together and stirred with one another for 5 minutes.
II Add component C to mixture I and likewise stir for 5 minutes.
III Weigh in components D together and then add to mixture II, subsequently stir for a further 5 minutes.
IV Add component E to mixture III and stir until component E has completely swollen (at least 2 hours).

Formulation Examples 5 to 7 are in accordance with the invention. For comparison, Formulation Examples C1 to C3 were prepared. Their compositions correspond to those of Formulation Examples 5 to 7 although, instead of the solubilizer as in Example 1, 2 or 3, Emulsogen® HCO 040 (C1), Genapol® UD 080 (C2) and Genapol® UD 088 (C3) was used. The exchange was made, based on the weight, in a ratio of 1:1.

To assess the performance of the solubilizers according to the invention in clear formulations, the transparency of the formulations was determined by means of NTU values. NTU is the abbreviation for Nephelometric Turbidity Unit. The calibration unit for turbidity measurements in the scattered-light method at 900 was applied in accordance with the directions of the US-EPA (Environmental Protection Agency), the standard being based on formazine solution.

The Nephelometric Turbidity Unit (NTU) is a unit used in water treatment for turbidity measurements in liquids. It is the unit of a turbidity of a liquid measured using a calibrated nephelometer.

TABLE 1
Transparency of the aftershave gels according to formulations 5 to 7

| | | Transparency of the |
|---|---|---|
| Formulation Examples | Solubilizer used | gels (NTU) |
| 5 (invention) | Example 1 | 27 |
| 6 (invention) | Example 2 | 16 |
| 7 (invention) | Example 3 | 21 |
| C1 (comparison) | Emulsogen ® HCO 040 | 36 |
| C2 (comparison) | Genapol ® UD 080 | 3042 |
| C3 (comparison) | Genapol ® UD 088 | 3970 |

### Formulation Examples 8 to 10

### Olive Oil Shower Gel

Composition:

| | |
|---|---|
| Solubilizer as in Example 1, 2 or 3 | 2.50% |
| Olive oil | 0.25% |
| Genagen ® 3 SB | 40.00% |
| Water | ad 100% |
| Genapol ® DAT | 7.00% |
| Phenonip ® | 0.50% |

Preparation:

The individual components are successively weighed in and mixed together with stirring.

Formulation Examples 8 to 10 are in accordance with the invention. Comparative Example C4 corresponds to the composition of Formulation Examples 8 to 10 but using Emulsogen® HCO 040 instead of the solubilizers as in Example 1, 2 or 3. The exchange was made, based on the weight, in a ratio of 1:1.

TABLE 2
Transparency of the shower gels according to formulations 8 to 10

| | | Transparency of |
|---|---|---|
| | | the shower gel |
| Formulation Examples | Solubilizer used | (NTU) |
| 8 (invention) | Example 1 | 25 |
| 9 (invention) | Example 2 | 11 |
| 10 (invention) | Example 3 | 21 |
| C4 (comparison) | Emulsogen ® HCO 040 | 43 |

### Formulation Examples 11 to 14

### Permanent Wave Formulation Containing Perfume Oil

Composition:

| A | Water | ad 100% |
|---|---|---|
| B | Ammonia solution (25%) | 8.2% |
| | Thioglycolic acid (80%) | 10.0% |
| | Ammonium hydrogencarbonate | 1.5% |
| | Genamin ® PDAC | 1.5% |
| C | Perfume oil | 0.8% |
| | Solubilizer as in Example 1, 2, 3 or 4 | 0.8% |

Preparation:
I Components B are added one after the other to component A with stirring.
II Components C are added together to mixture I and, during this, everything is stirred together.

Formulation Examples 11 to 14 are in accordance with the invention. Example C5 serves for comparison. It corresponds to the composition of Formulation Examples 11 to 14 but using Emulsogen® HCO 040 instead of the solubilizers as in Example 1, 2, 3 or 4. The exchange was made, based on the weight, in a ratio of 1:1.

TABLE 3
Transparency of permanent wave formulations 11 to 14

| | | Transparency of the |
|---|---|---|
| Formulation | | permanent wave |
| Examples | Solubilizer used | formulations (NTU) |
| 11 (invention) | Example 1 | 20 |
| 12 (invention) | Example 2 | 17 |
| 13 (invention) | Example 3 | 19 |
| 14 (invention) | Example 4 | 24 |
| C5 (comparison) | Emulsogen ® | 351 |
| | HCO 040 | |

### Examples 15 to 26

### Dissolving Power of Individual Substances (Rosemary Oil, Vanilla and Menthol) in Aqueous Solution

Composition of the test mixtures

| | |
|---|---|
| Test substance | x % |
| Solubilizer | 10% |
| Water | ad 100% |

Procedure:

To test the dissolving power, various amounts x of the test substances were stirred with solubilizer at 800 revolutions for 5 minutes and then the corresponding amount of 65° C.-hot water was added. After stirring for a further 5 minutes, the clarity of the solution is evaluated.

Examples 15 to 26 are in accordance with the invention. Examples C6 to C8 serve for comparison.

TABLE 4
Dissolving power (quantitative data in % by weight)

| | | | Use amount of |
|---|---|---|---|
| | | | the test |
| | | | substance until |
| Formulation | Solubilizer used | | clouding of the |
| Examples | [10%] | Test substance | test mixture [x %] |
| 15 (invention) | Example 1 | Rosemary oil | 3 |
| 16 (invention) | Example 2 | Rosemary oil | 3 |
| 17 (invention) | Example 3 | Rosemary oil | 3 |
| 18 (invention) | Example 4 | Rosemary oil | 3 |
| C6 (comparison) | Genapol ® | Rosemary oil | 1 |
| | UD 080 | | |
| 19 (invention) | Example 1 | Vanilla | 6 |
| 20 (invention) | Example 2 | Vanilla | 6 |
| 21 (invention) | Example 3 | Vanilla | 7 |
| 22 (invention) | Example 4 | Vanilla | 6 |
| C7 (comparison) | Emulsogen ® | Vanilla | 5 |
| | HCO 040 | | |
| 23 (invention) | Example 1 | Menthol | 3 |
| 24 (invention) | Example 2 | Menthol | 4 |
| 25 (invention) | Example 3 | Menthol | 4 |
| 26 (invention) | Example 4 | Menthol | 4 |
| C8 (comparison) | Emulsogen ® | Menthol | 1 |
| | HCO 040 | | |

Chemical name of the commercial products used:

| Aristoflex ® AVC | ammonium acryloyldimethyltaurate/VP copolymer |
|---|---|
| Emulsogen ® HCO 040 | hydrogenated castor oil with 40 EO |
| Emulsogen ® HCO 060 | hydrogenated castor oil with 60 EO |
| Genagen ® 3 SB | mixture of cocobetaine, sodium |
| | methylcocoyltaurate and sodium cocoylisethionate |
| Genamin ® PDAC | polydiallyldimethylammonium chloride |
| Genapol ® DAT | stearyl ester of polyglycol ethoxylated with 150 EO |
| Genapol ® UD 080 | mixture of linear and branched C₁₁-oxo alcohol polyglycol |
| | ether with 8 EO (weight ratio linear:branched = 50:50) |
| Genapol ® UD 088 | mixture of linear and branched C₁₁-oxo alcohol polyglycol |
| | ether with 8 EO (80% by weight) and water (20% by weight) |
| | (weight ratio linear:branched = 50:50) |
| Genapol ® UD 110 | mixture of linear and branched C₁₁-oxo alcohol polyglycol |
| | ether with 11 EO (weight ratio linear:branched = 50:50) |
| Phenonip ® | preservative blend of phenoxyethanol, methylparaben, |
| | ethylparaben, butylparaben propylparaben, |
| | isobutylparaben |
| Polyglycol 400 | polyethylene glycol with an average molecular weight of |
| | 400 |
| Polyglycol 35000 p | polyethylene glycol with an average molecular weight of |
| | 350 000 |

“EO” means a structural unit derived from ethylene oxide, i.e. a structural unit —CH₂CH₂O—.

The commercial products used are sold by Clariant.

1. A solubilizer comprising
a) at least one ethoxylated fatty alcohol according to formula (I) 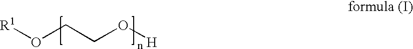
wherein R¹ is a linear or branched alkyl radical having 6 to 12 carbon atoms and n is on average a number from 2 to 12,
b) at least one ethoxylated triglyceride whose acyl radicals are derived from linear or branched saturated fatty acids having 6 to 22 carbon atoms, and
c) water,
wherein the solubilizer is liquid and clear at room temperature and does not contain addition products of ethylene oxide and simultaneously propylene oxide onto fatty alcohols.

2. The solubilizer as claimed in claim 1, which comprises at least two ethoxylated fatty alcohols according to formula (I) and wherein the weight ratio of ethoxylated fatty alcohols of the formula (I) with linear radicals R¹ to ethoxylated fatty alcohols of the formula (I) with branched radicals R¹ is from 40:60 to 60:40.

3. The solubilizer as claimed in claim 1, wherein R¹ is a linear or branched alkyl radical having 11 carbon atoms.

4. The solubilizer as claimed in claim 1, wherein n on average is a number from 6 to 9.

5. The solubilizer as claimed in claim 1, wherein the at least one ethoxylated triglyceride of component b) is selected from addition products of, on average, 20 to 100 mol, of ethylene oxide onto at least one triglyceride.

6. The solubilizer as claimed in claim 1, wherein the at least one ethoxylated triglyceride of component b) is selected from addition products of, on average, 20 to 100 mol, of ethylene oxide onto hydrogenated castor oil.

7. The solubilizer as claimed in claim 1, which comprises the at least one ethoxylated fatty alcohol of component a) in an amount of from 20 to 70% by weight, the at least one ethoxylated triglyceride of component b) in an amount of from 20 to 60% by weight, and water in an amount of from 1 to 20% by weight.

8. The solubilizer as claimed in claim 1, which is free from components containing propylene oxide.

9. The solubilizer as claimed in claim 1, which consists of the at least one ethoxylated fatty alcohol of component a), the at least one ethoxylated triglyceride of component b) and water.

10. A cosmetic, dermatological or pharmaceutical formulation comprising at least one solubilizer as claimed in claim 1.

11. A method for producing a cosmetic, dermatological or pharmaceutical formulation comprising the step of adding a solubilizer as claimed in claim 1, wherein the solubilizer comprises
a1) C₁₁-alkyl-O—(CH₂CH₂O)₈—H,
b1) ethoxylated hydrogenated castor oil with 40 ethylene oxide units, and
c) water and
d) either does not comprise substances containing propylene oxide or if it does comprise substances containing propylene oxide, comprises, based on the finished formulation, 1.0% by weight or less, of substances containing propylene oxide,
to at least one other component of a cosmetic, dermatological or pharmaceutical formulation.

12. A cosmetic, dermatological or pharmaceutical formulation which comprises
a1) C₁₁-alkyl-O—(CH₂CH₂O)₈—H,
b1) ethoxylated hydrogenated castor oil with 40 ethylene oxide units, and
c) water and
d) either does not comprise substances containing propylene oxide or if it does comprise substances containing propylene oxide, comprises, based on the finished formulation, 1.0% by weight or less, of substances containing propylene oxide.

13. The formulation as claimed in claim 11, which comprises, based on the finished formulation, the ethoxylated alcohol of component a1) from 0.05 to 6.0% by weight and the ethoxylated hydrogenated castor oil with 40 ethylene oxide units of component b1) from 0.02 to 5.0% by weight.

14. The formulation as claimed in claim 11, which does not comprise substances containing propylene oxide.

15. The formulation as claimed in claim 11, which comprises, based on the finished formulation, an amount of water of from 30.0% by weight or more.

16. The formulation as claimed in claim 11, which comprises components that are sparingly soluble in water.

17. The formulation as claimed in claim 16, which comprises, based on the finished formulation, 0.01 to 30.0% by weight, of components that are sparingly soluble in water.

18. The formulation as claimed in claim 1, which has a clear appearance.
